(19) United States
(12) Patent Application Publication
(10) Pub. No.: US 2003/0032032 A1
(43) Pub. Date: Feb. 13, 2003

(54) Early pre-symptomatic prion diagnostic blood test for encephalopathies

(76) Inventors: Annelies Resink, Paris (FR); Nathalie Fuentes, Kremlin Bicetre (FR); Fabien Schweighoffer, Vincennes (FR)

Correspondence Address: NIXON & VANDERHYE P.C., 1100 North Glebe Road, 8th Floor, Arlington, VA, 22201-4714, US

(21) Appl. No.: 10/100,178

(22) Filed: Mar. 19, 2002

Related U.S. Application Data
(60) Provisional application No. 60/278,670, filed on Mar. 21, 2001.
(60) Provisional application No. 60/282,463, filed on Apr. 10, 2001.

(51) Int. Cl. C12Q 1/68

(52) U.S. Cl. 435/6

(57) ABSTRACT

This invention relates to compositions and methods of detecting encephalopathies in a subject. This invention also relates to genetic markers, nucleic acid preparations or libraries, and kits for use in the implementation of said detection methods. The compositions and methods of this invention can also be used for the diagnosis, characterization, progression monitoring, etc. of encephalopathies, including at early stages thereof, particularly Transmissible Spongiform Encephalopathies (TSE), including Bovine Spongiform Encephalopathies (BSE, “Mad Cow disease”).

This application claims benefit of, and incorporates by reference, U.S. Provisional Application Nos. 60/278,670, filed Mar. 21, 2001, and 60/282,463, filed Apr. 4, 2001.

This invention relates to compositions and methods of detecting encephalopathies in a subject. This invention also relates to genetic markers, nucleic acid preparations or libraries, and kits for use in the implementation of said detection methods. The compositions and methods of this invention can also be used for the diagnosis, characterization, progression monitoring, etc. of encephalopathies, including at early stages thereof, particularly Transmissible Spongiform Encephalopathies (TSE), including Bovine Spongiform Encephalopathies (BSE, “Mad Cow disease”).

Encephalopathies, more particularly Transmissible Spongiform Encephalopathies (TSEs) consist of a unique group of invariably fatal neurological disorders, which affect both human and animals and which are characterised by long pre-symptomatic incubation periods of months or years, and brain lesions associated with deposits of protease-resistant proteins. The nature of the infectious agent has not been definitively determined, although the predominant theory is that a previously unrecognised pathogenic agent called a prion, an abnormally folded protein, is responsible.

One of the most common form is Bovine Spongiform Encephalopathies (BSE), which affects cows and cause the “Mad Cow” disease. There is a new urgency in the efforts to determine the scale of the BSE epidemic and to safeguard public health. The EU (European Union) agreed last December to the systematic BSE diagnostic testing of all slaughtered cattle older than 30 months. Since BSE-incubation time in cattle is around five years, during which infection can probably be spread by lateral and vertical transmission, the development of an early pre-symptomatic test in living animals is of vital importance. Such a pre-clinical diagnostic test will offer a means to reliably exclude infected animals from the human food chain. Furthermore, the infectious BSE agent can infect sheep and goats, including genotypes resistant to the sheep-specific TSE agent. This latter observation signals a need for pre-clinical testing program of BSE in sheep flocks in order to prevent further human food contamination. So far, the only test available to identify the presence of BSE infection prior to clinical manifestations of the disease is a bioassay consisting of the injection of contaminated brain tissue into mice followed by the observation of disease development. Because this bioassay takes months to finish, it is an impractical tool for systematic testing.

As of November 2000, a total of 180,000 cows were found to be infected in United Kingdom and an additional 1,500 in Ireland, Portugal, Switzerland, Germany, Italy, Spain and France. Approximately 320,000 diagnostic tests have been performed to date using three products (from three companies) approved by the EU. The average cost per test is $23 (ranging from $15 to $30), not including the cost of obtaining the brain tissue sample. The EU is evaluating five other BSE tests, but like the three tests that are already approved, they cannot be performed until the animal is slaughtered. The EU has ordered that mandatory BSE testing begin in July 2001 for seven million slaughtered cows annually and it is expected that a total of 10 million tests will be sold and administered over the coming year.

There is thus a need for new methods of detecting encephalopathies, particularly methods that can be performed on living animals, are rapid, and preferably, can detect the pathology at pre-symptomatic stage. It is the object of this invention to provide such a pre-symptomatic blood test for encephalopathies, particularly for TSE, including BSE, in a mammal. The invention allows to readily test potentially every animal at risk, optionally multiple times, during the life of the animal. This invention also relates to genetic markers, nucleic acid preparations or libraries, and kits for use in the implementation of said detection methods.

Applicants have created a pre-symptomatic diagnostic test in easily accessible body fluids of living animals. Applicants have undertaken an extensive research and development program using an innovative approach to identify new markers for TSE. These and other aspects represent objects of the present application.

This invention thus relates to a method of detecting the presence of an encephalopathy in a subject, the method comprising (i) collecting or providing a biological sample containing nucleic acids from the subject, typically a fluid sample (e.g., blood, serum, saliva, urine, etc.), although other tissue or cell sample may be used as well, and (ii) contacting said sample with at least a nucleic acid molecule comprising the sequence of all or part of a sequence selected from SEQ ID Nos 1-15 or a sequence complementary thereto, under conditions allowing hybridisation to occur, and (iii) determining the presence of such hybrids, the presence of hybrids indicating the presence of an encephalopathy in the subject.

This invention also relates to a method of determining or detecting subject (e.g., a mammal) at risk of developing an encephalopathy, the method comprising (i) collecting or providing a biological sample containing nucleic acids from the mammal, typically a fluid sample (e.g., blood, serum, saliva, urine, etc.), although other tissue or cell sample may be used as well, and (ii) contacting said sample with at least a nucleic acid molecule comprising the sequence of all or part of a sequence selected from SEQ ID Nos 1-15 or a sequence complementary thereto, under conditions allowing hybridisation to occur, and (iii) determining the presence of such hybrids, the presence of hybrids indicating a risk of developing an encephalopathy in the mammal.

The nucleic acid may be immobilized on a support, such as a chip, filter, membrane, glass slide, etc. The contacting step may comprise any combination of the above sequences and, typically, uses at least two, preferably at least 3.

In a first variant, the contacting step above comprises contacting the sample with at least a nucleic acid molecule comprising the sequence of all or part of SEQ ID NO: 1 or a functional equivalent thereof or a sequence complementary thereto.

SEQ ID NO:1
NE206922 Preproelastase
CCCCAGAGAGGAGCCGAAGCTCACGATGCCATGCACCTGCCACTGGCCATTAGATGCCCG
GCAATTCAGTGGTCCGCCAGAGTCCCCATTGCAGCTGGAGGTCACGCCGTCGCCACCAGC
GCACACCATGCTGGACTTCACAGAGCTTCCCCACCAGCTAGCGCTGGAGCAGGTGGCATA
GTCCACAACCAGCAGGCGGCCCTGCCTCAGGGTGTCAGGACTGTTCCCATTGGTCTGCAG
CAGGCCCCAGCCTGTGACATAGCAGACATAGTTTCTCGGGAGAATGGTGCCAGCGGGTGG
GAGGCAAGCTGTCTGGAT

In a further variant, the contacting step above comprises contacting the sample with at least a nucleic acid molecule comprising the sequence of all or part of SEQ ID NO:2 or a functional equivalent thereof or a sequence complementary thereto.

SEQ ID NO:2
NE206230 Chymotrypsin-like protease
ATGGACCAGGGGTAGAACAACTTGCTGCAGGCGAGCTGGTGTCACATTGCCCACACCACT
GATTCGGCCCCAGCCAGTGGTGACACAGGTGAGCCCCGAAGGCAGTGCCTCGTTTGTGGA
AGCCAGGCAGACTGGTGAGACTTGTGCTGTGTACCGGGCTGGCGAGGCAAGCTTCAGGAG
AGTCAGGTCATTGTTCATGGTGTTGGCGTTCCAGTTAGGGTGGC

In a further variant, the contacting step above comprises contacting the sample with at least a nucleic acid molecule comprising the sequence of all or part of SEQ ID NO:3 or a functional equivalent thereof or a sequence complementary thereto.

SEQ ID NO:3
NE212490 Unknown
CGGTGGCAAGTCGGGTTCCAGGTCCATGAAGCCCCCGGGAGGAGAATCGAGCGATCTTTT
CGGAAGTCCAGAAGAAGGTATTTCTTCAAGCAAGCCTAATAGGATGGCATCTAATATTTT
CGGACCAACTGAAGAACCTAAAAACATACCCAAGAGGACAAATCCTCCAGGAGGCAAAGG
AAGTGGGATCTTTGATGAATCGACTCCTGTGCAAACTCGACAACGTTTGAATCCACCAGG
GGGGAAGACCAGTGACATATTTGGGTCCCCAGTCCT

In a further variant, the contacting step above comprises contacting the sample with at least a nucleic acid molecule comprising the sequence of all or part of SEQ ID NO:4 or a functional equivalent thereof or a sequence complementary thereto.

SEQ ID NO:4
NE212911 Chymotrypsinogen
CAACAGACACAGTCTCAGAGAACTGGGCAGGAGTGGCCAGCTTCAGCAGGGTGATGTCAT
TACGCACGGTGAAGGAGTTGAACTTGGGGTTCTTAAAAACCTGAGCGATTTTCAGGACCT
GGACATTCTCTTCGTCGGAGCCCTGATCAAACTCTCCAGCTACCACCACATCGGTTGTCT
TGACCCCGCAGTGGGCAGCAGTGACCACCCAGTTTTCGCTGATGAGGGAGCCCCCGCAGA
AATGGAAGCCAGTTCTGTCCTGCAGGGACACCTGCCAGGGCCAGGAGCCAGGGATAGCAT
CCT

In a further variant, the contacting step above comprises contacting the sample with at least a nucleic acid molecule comprising the sequence of all or part of SEQ ID NO:5 or a functional equivalent thereof or a sequence complementary thereto.

SEQ ID NO:5
NE211662 Amylase-2
GTCTTGGTGGTCCAATCCAGTCATTCTGATCTTTTCCTTCTGGAAATTTCTATTCCAACG
GTAACTCGACATTACTCTTGTGAATCCATAAGGATGAGCCNTCATAAATCCGACAGCCAT
TTTATACATT

In a further variant, the contacting step above comprises contacting the sample with at least a nucleic acid molecule comprising the sequence of all or part of SEQ ID NO:6 or a functional equivalent thereof or a sequence complementary thereto.

SEQ ID NO:6
NE216391 Kinase substrate HASPP28
GGGTGGCCTCGAACTCAGAAATCCGCCTGCCTCTGCCTCCCGAGTGCTGGGATTAAAGGC
GTGGCCACCACGCCCGGCTTTGCATGCTTTATTTCTTGTGGAATAACTGACACCCAAGTT
CTCCTTCAGAAGCTTCAGCCAAGCCCACCTTGAGGAACAAGACGAGGACACATGATGGGT
GAGACATGGCAGAGGTCCTGGCGGCACGGCCCAGTTCCCCGGCATCTCCTCCCACAGGCC
AGCTACTTATTCAGGGACAGCGACTG

In a further variant, the contacting step above comprises contacting the sample with at least a nucleic acid molecule comprising the sequence of all or part of SEQ ID NO:7 or a functional equivalent thereof or a sequence complementary thereto.

SEQ ID NO:7
NE208331 Carboxyl ester lipase
CGTGGACGATGGCTAAGTACCGGTCTACAGCTATACAGGCCAGCAGCAGGCTCTGCAGT
AGAAATTGATCTTGTGCAGAGCGATCACAGTTTTGCAGAGGAAGGTCCCTAGGACCCAAC
CCACAGAGCCCTCAGCCACTGCAAAAGGCAGGATGAAGACTAAGAGAAGGTCGGCTACTG
CGAGGTGGAACAGGAAGGTCTCGGTTGAGCTCCGCGTGTGCCGGTGCCTCTCCGGGATTC
CAGCACCAGGATGTTTCCCATCATACCCAGGAGGAAGATGAGGCTGTAGGCCCAGGCATG
AATCCGCCTTAAGGACGTCAGTAAGGGTCCCTCGACTGTAGAGCAGAAGTTCTGTCTGTA
GG

In a further variant, the contacting step above comprises contacting the sample with at least a nucleic acid molecule comprising the sequence of all or part of SEQ ID NO:8 or a functional equivalent thereof or a sequence complementary thereto.

SEQ ID NO:8
NE228092 Unknown
GGATGCATTTGAACTGATAAGGACTAGGTAGAACTGAAGGGCTAGATGGAATGTTACGGC
CTAGGTATAACGTTAAGCCTAAGTAACTCTTACGTGGCTAGCCTGCCATTTTGCGCTGTT
ACTAGTATTATAAGGAAACTTCCTTATGTGCAAGTTGATTGCATATTCTCTTAAATTCTT
TGCTCTTGGAAACTGAGCACAACAGAGGTTAGTTAGAACTGCTCTGTATAGTTAGCCAAA
ATGAGCTTTGACCCAATCAGCCAATCAGCAGCACTTCTGCATATGTGTAAAGCTTGTATG
GTATCTGCTTTTATAAGCTG

In a further variant, the contacting step above comprises contacting the sample with at least a nucleic acid molecule comprising the sequence of all or part of SEQ ID NO:9 or a functional equivalent thereof or a sequence complementary thereto.

SEQ ID NO:9
NE230511 Unknown
GTGTCCAGGAGGGAACTGGTATGATCTAATGAATCCTTTTACTAAGATGGGGATGTGATG
GTAGCACACAGCAGGGAAGAGGGACTTCGAATCTCAGGCCTCAGCTTAGAAGGGGAAGCA
CCTATTTCCACTGCCCCTTCTTTAAGACATCTCCCTTTTGCTGAGGCTTACCAGGGGGTA
GGGGAGCGCAGGGAAGGTCAAGGAGGTGTATCAAAGTATC

In a further variant, the contacting step above comprises contacting the sample with at least a nucleic acid molecule comprising the sequence of all or part of SEQ ID NO:10 or a functional equivalent thereof or a sequence complementary thereto.

SEQ ID NO:10
NE230512 Unknown
AGACACTTTGATACACCTCCTTGACCTTCCCTGCGCTCCCCTACCCCCTGGTAAGCCTCA
GCAAAAGGGAGATGTCTTAAAGAAAGGGGCAGTGGAAATAGGTGCTTCCCCTCCTAAGCT
GGGGCCTGAGATTCGAAGTCCCTCTTCCCTGCTGTGTGCTACCATCACATCCCCATCTTA
GTAAAAGGATTCATTAGATCATACCAGTTCCCTCCTGGACACCC

In a further variant, the contacting step above comprises contacting the sample with at least a nucleic acid molecule comprising the sequence of all or part of SEQ ID NO:11 or a functional equivalent thereof or a sequence complementary thereto.

SEQ ID NO:11
NE230551 Unknown
TACCATGAGGGAGTGGCTGGATTAGGCCTAGGGAGGATGACTGTCCATGAGAGATGACAG
GTGTGGGCAGCTCTTCTAGGGGGTGTGGGCACTGGAGTAGCCTCAGGAGGCAGGCTCC
CCCGCTGTTGGTTCTGAGACTGGTGAGGCGGGACCAGCCCCGTTGTTTCCAGTTCTTCAT
GCCTGGTGGCACCCTCA

In a further variant, the contacting step above comprises contacting the sample with at least a nucleic acid molecule comprising the sequence of all or part of SEQ ID NO:12 or a functional equivalent thereof or a sequence complementary thereto.

SEQ ID NO:12
NE230612 Unknown
GGGTAAAAGAGGGAAATGAAAAGGAGAGAGACAGTATCCAGCTCGGTAAACAGTTTCCCT
AAGTGTTCTCCACCATGTGGAACACACAGGAGATTCATGGGAGTTGGGTAGAGAAGAGAA
GGGGGAAGGAGGAGACAGAGGCA

In a further variant, the contacting step above comprises contacting the sample with at least a nucleic acid molecule comprising the sequence of all or part of SEQ ID NO:13 or a functional equivalent thereof or a sequence complementary thereto.

SEQ ID NO:13
NE213890 G-protein beta2 subunit
CTGACCCAGATCACAGCCTGGGCTGGTACCCAGTGGGGCGAATTCAGATGAGTAACACGG
AGGACCCTCCGTGGACACCTGGCAAAAATCTATGCCATGCACTGGGGGACAGACTCAAGG
CTGCTGGTCAGCGCCTCCCAGGACGGAAAGCTCATCATTTGGGACAGCTCACCACTAACA
AGGTCCACGCCATCCCTCTGCGTTCCTCCTGGGTAATGACCTGTGCCTCGCCCCTCAGG
GAACTTTGTGGCCTGTGGGGGTTTGGACAACATCTGCTCCATCTATAGTCTCAAGACCCG
AGAGGGCAAT

In a further variant, the contacting step above comprises contacting the sample with at least a nucleic acid molecule comprising the sequence of all or part of SEQ ID NO:14 or a functional equivalent thereof or a sequence complementary thereto.

SEQ ID NO:14
NE214232 Mitochondrion
TCGACCCCCGCCTGTTTACCAAAAACATCACCTCTAGCATTACAAGCTATTAGAGGCACT
GCCTGCCCAGTGACTAAAGTTTAACGGCCGCGGTATCCTGACCGTGCAAAGGTAGCATAA
TCACTTGTTCCTTAATTAGGGACTAGCATGAACGGCTAAACGAGGGTCCAACTGTCTCTT
ATCTTTAATCAGTGAAATTGACCTTTCAGTGAAGAGGCTGAAATATAATAATAAGACGAG
AAGACCCTATGGAGCTAAATTATATAACTTATCTATTAATTTATAAACCTAATGGCCCAA
AACTAT

In a further variant, the contacting step above comprises contacting the sample with at least a nucleic acid molecule comprising the sequence of all or part of SEQ ID NO:15 or a functional equivalent thereof or a sequence complementary thereto.

SEQ ID NO:15
NE21161O Exostoses multiple 2 (EXT2)
TGGCTGTGTCCCAGTTGTCATTGCAGACTCTTATATTCTGCCTTTCTCTGAAGTTCTGGA
CTGGAAGAGGGCATCTGTGGTCGTTCCAGAGGAAAAGATGTCAGATGTGTACAGCATCCT
GCAGAACATCCCACAGAGGCAGATTGAAGAGATGCAGAGACAGGCACGGTGGTTCTGGGA
GGCATACTTCCAGTCCATTAAAGCCATTGCCCTGGCCCCCTACAGATCATCAATGACAGG
ATCTATCCATATGCAGCCTCTCCTATGAAGAGTGGAATGACCCTCCTGCTGTGAAGTGGG
CTA

Preferably, for screening purposes, the biological sample is treated to render nucleic acids or polypeptides available for detection (e.g., for hybridization or antigen-antibody reaction). Treatment may include cell lysis, particularly using chemical, mechanical or physical means. Furthermore, the nucleic acids in the sample may be labeled prior to hybridization, for instance by conventional radiolabels, fluorescent labels, enzymatic labels, chemoluminescent labels, etc. Hybridization can be performed under any conventional techniques and conditions, which are known to the skilled person and can be adjusted by the skilled person. In this regard, the hybridization can be carried out under high, intermediate or low stringency, depending on the desired level of sensitivity, quantity of available material, etc. For instance conditions suitable for hybridization include a temperature of between about 62 and 67° C. for 2 to 18 hours. Following hybridization, various washes may be performed to remove non-hybridized molecules, typically in SSC buffers comprising SDS such as 0.1 to 10×SSC, 0.1% SDS.

In a typical experiment, the nucleic acids (or arrays or chips or filters) are prehybridized in hybridization buffer (Rapid Hybrid Buffer, Amersham) containing 100 μg/ml of salmon sperm DNA at 65° C. for 30 min. The nucleic acids from the sample are then applied to the filter (0.5×10⁶ to 1×10⁶ cpm/ml) at 65° C. for 2 to 18 hours. Filters are washed in 5×SSC buffer, 0.1% SDS at 65° C. for 30 min then in 0.2×SSC buffer, 0.1% SDS. The hybridization profiles are analyzed according to known techniques, for example by measuring the radioactivity with an InstantImager (Packard Instruments). The hybridization conditions may be adjusted by those skilled in the art according to conventional techniques, particularly by decreasing the hybridization temperature and/or by increasing the salt concentration of the hybridization buffer.

The invention also relates to various genetic markers of encephalophaties, particularly TSEs. These markers have been identified from infected mammals and can be detected in biological fluids, including blood, serum, saliva, urine, etc., i.e., with no need to perform tissue biopsies. The markers more specifically represent qualitative genetic differences between healthy and affected mammals. These markers have been prepared using the DATAS technology disclosed in WO99/46403, incorporated therein by reference. DATAS identifies qualitative differences between expressed genes and provides a systematic analysis of alternative RNA splicing events between two conditions: either healthy/diseased, untreated/treated or control/infected. Thus, DATAS leads to the identification of functionally distinct RNA variants and thus also proteins, which play a role in cellular equilibrium. The technique involves three different steps including tissue collection, RNA isolation and construction of a database of events showing qualitative differences. Identifying qualitative differences via DATAS clearly holds a stronger interest for diagnostics than identifying sequences up or down regulated through the use of classical genomic profiling approaches. DATAS-based qualitative differences represent new sequence fragments not present in previous expression profiles that can be selected for characterising a given patho-physiological situation.

Several different signatures (or genetic markers) that are present specifically in the blood from affected mammals have been isolated, as described in the examples.

These genetic markers more precisely comprise all or part of any one of nucleic acid sequences SEQ ID Nos 1 to 15, or functional equivalents thereof.

This invention thus relates also to a nucleic acid molecule selected from the group of SEQ ID Nos 1-15 or a fragment thereof, a sequence complementary thereto or a functional equivalent thereof.

This invention also relates to a vector comprising a nucleic acid as described above, as well as to recombinant host cells comprising such a nucleic acid molecule or vector.

Another object of this invention lies in the use of a nucleic acid molecule comprising the sequence of all or part of a sequence selected from SEQ ID Nos 1-15 or a sequence complementary thereto or a functional equivalent thereof, for the detection of a pathological event in a subject, more preferably of the presence of an encephalopathy.

Within the context of this invention, the term “functional equivalent of a sequence” designates any nucleic acid molecule that can hybridise with or detect said sequence or a complementary strand thereof, as well as any nucleic acid molecule that can hybridise with or detect a gene, RNA or genetic deregulation event (e.g., splicing, rearrangement, mutation, etc.) in a gene or RNA, that is detected by said sequence. In other words, the present invention discloses the identification of target genes and the methods or compositions of this invention include any nucleic acid sequence that can detect said gene or deregulation event in a sample. Such target genes include for instance a preproelastase gene or RNA comprising SEQ ID NO: 1, a chymotrypsin-like protease gene or RNA comprising SEQ ID NO: 2, a chymotrypsinogen gene or RNA comprising SEQ ID NO: 4, an amylase-2 gene or RNA comprising SEQ ID NO: 5, a kinase substrate HASPP28 gene or RNA comprising SEQ ID NO: 6, a carboxyl ester lipase gene or RNA comprising SEQ ID NO: 7, a G-protein beta2 subunit gene or RNA comprising SEQ ID NO: 13, an exostoses multiple 2 gene or RNA comprising SEQ ID NO: 15, as well as any gene or RNA comprising SEQ ID NO: 3, 8-12 and 14. Functional equivalents may thus comprise a sequence that overlaps with one of the above sequences, or is specific for a distinct region in the gene or RNA, or for a distinct genetic alteration in the gene or RNA. Functional equivalents also include (i) corresponding nucleic acids from different species as well as (ii) nucleic acid sequences having one or several sequence variation(s) such as mutation(s), substitution(s) addition(s) or deletion(s) of one or several bases, and retaining substantially the same specificity. Preferably, sequence variations do not affect more than 5% of the sequence.

The nucleic acid molecule may include all or part of the sequence disclosed, and may comprise additional sequence corresponding to synthetic sequence (e.g. cloning sites) or to flanking sequence in the target gene or RNA. The nucleic acid may be a DNA (e.g., cDNA, gDNA), RNA, oligonuleotide, PCR fragment, probe, etc. It may be single-stranded or double-stranded.

Within the context of the present invention, a “part” of the above nucleic acid sequences includes any fragment of said sequences comprising at least 5 consecutive bases, more preferably at least 7 consecutive bases, even more preferably at least 8 consecutive bases. Indeed, the fragment or part should be sufficiently long to exhibit the selectivity of the entire sequence in a hybridisation experiment under high stringency. Preferred part include at least 10 consecutive bases, typically at least 15 consecutive bases.

A sequence complementary to the above sequences designates any sequence having full complementarity therewith or only partial complementarity. Partial complementarity indicates that certain mismatches would be tolerated, as long as the nucleic acid retains a specificity in hybridisation experiments. For instance, a mismatch every 15 bases would not substantially alter the ability of a nucleic acid molecule to retain the hybridisation profile.

The invention preferably uses nucleic acid molecules of between about 10 and about 800 bases in length, specific for a gene as described above, for detecting encephalopathies in a sample.

The invention also includes vectors comprising a nucleic acid as defined above. The vector may be a plasmid, episome, chromosome, phage, virus, etc. The vector may comprise regulatory sequences, such as a promoter, origin of replication, selection gene, polyA sequence, secretion sequence, etc. Typical examples of plasmids include commercially available plasmids such as pBR, pUC, pcDNA, etc. Suitable examples of viruses include replication defective adenoviruses, retroviruses, AAVs or herpes viruses.

Recombinant host cells comprising a nucleic acid or a vector as defined above include prokaryotic or eukaryotic cells, such as bacteria (e.g., E. coli), yeasts (e.g., Saccharomyces, Kluyveromyces, etc.), plant cells, insect cells, mammalian cells, etc. Mammalian cells may be derived from various species, including rodents, bovines, monkey and humans. They may be primary cells or established cell lines. Such cells include, for instance, CHO, COS, 3T3, HeLa, etc.

The compositions and methods of this invention can be used for the diagnosis, characterization, progression monitoring, etc. of encephalopathies, including at early stages thereof, particularly Transmissible Spongiform Encephalopathies (TSE), including Bovine Spongiform Encephalopathies (BSE, “Mad Cow disease”). The invention is also suitable to detect vCJD in human beings.

The value of having a pre-symptomatic blood test is, inter alia:

Identify the infected animal, and thus avert contact and subsequent infection with other members of the herd; current tests that rely on detection of the prion protein after slaughter can only detect the symptomatic stage of the disease;

Keeps sick animals out of the slaughter-house; currently slaughter houses are full of infected animals, detected and undetected;

Avoid complicated slaughter-house processing and tracking of animals, and avoid errors of mixing contaminated meat with meat approved for market;

The blood test can be carried out a few days or a week before bringing animals to the slaughter house, while the current test creates a bottle-neck at the slaughter-house;

Avoid the expense of brain tissue extraction and processing;

Detect different stages of the disease—early and late forms;

Animals found negative by the test can be re-tested to increase the opportunity to detect the disease, if present; and

Prevent the social and economic impact of killing entire herds; farmers just do not want to see their herds killed without solid scientific evidence or demonstration that such extreme measures are necessary and warranted.

The disclosed diagnostic methods do not require knowledge, with certainty, of the infectious agent as it is based on identified markers of the presence of the disease and the progression of the disease from early to late stages.

The methods are advantageous since they can be used to test for early, pre-symptomatic BSE in animals incubating the disease, and since they work from a simple blood samples.

The invention describes genetic markers from circulating fluids, isolated from test animals that are suspected to participate in the disease progression, and thus to be encoded by genes critical for the progression of the disease, and further capable of distinguishing the early and late stages of the disease.

Studies were performed in several mammalian species:

Reliable TSE-infected signatures have been detected in said species;

Genetic markers for the early, pre-symptomatic phase and symptomatic phases of the disease have been identified.

We have identified 5 signatures that are present in the blood of infected sheep;

In 4 individual infected sheep studied, these 5 signatures are present in the blood and are up-regulated in comparison to 2 control sheep;

In mice, we have identified 7 signatures that are present in the spleen of infected mice and are up-regulated in comparison to control mice;

The progression of the disease was studied over multiple timepoints:

signatures were followed over different timepoints from early stage to late stage of the disease;

3 signatures were highly expressed (up-regulated) at 35 days (pre-symptomatic early stage) and the expression diminished by 200 days (symptomatic late stage);

The new diagnostic test is thus based on a genome-wide analysis of differential expression of splice variants that occur between infected and uninfected individuals.

By applying a unique gene profiling technology, DATAS (Differential Analysis of Transcripts with Alternative Splicing), Applicants have now identified genetic markers for TSE infection. From these data we selected those signatures of downstream events that are induced or inhibited by the TSE infectious agent. Based on the large number of events that have been screened and are being validated it is likely that this diagnostic test will have greater impact and value than the available prion-antibody-based analysis currently being used to address the epidemic.

The invention also relates to the polypeptides encoded by the above nucleic acid molecules, and their use for diagnostic or therapeutic purposes. More specifically, an object of this invention resides in a polypeptide, wherein said polypeptide has an amino acid sequence encoded by a nucleic acid molecule as defined above.

The invention also relates to antibodies (monoclonal or polyclonal) directed against said polypeptides, as well as fragments or derivatives of said antibodies (e.g. Fab, Fab′2, ScFv, humanized antibodies, etc.). Such antibodies may be produced according to conventional methods, including immunization of an animal and collection of serum (polyclonal) or spleen cells (to produce hybridomas by fusion with appropriate cell lines). Methods of producing polyclonal antibodies from various species are well known in the art. As an example, the antigen may be combined with an adjuvant (e.g., Freund's adjuvant) and administered to an animal, typically by sub-cutaneous injection. Repeated injections may be performed. Blood samples are collected and immunoglobulins or serum are separated. Methods of producing monoclonal antibodies from various species are also known in the art (Harlow et al., Antibodies: A laboratory Manual, CSH Press, 1988). Briefly, these methods comprise immunizing an animal with the antigen, followed by a recovery of spleen cells which are then fused with immortalized cells, such as myeloma cells.

The resulting hybridomas produce the monoclonal antibodies and can be selected by limit dilutions to isolate individual clones. Preferred antibodies of this invention are antibodies that specifically bind an epitope comprised in the polypeptides encoded by SEQ ID NOs: 1 to 15.

These antibodies can be used for therapeutic or diagnostic purposes. In particular, the test may be based on the detection of the above polypeptides or parts thereof in a biological sample, using said antibodies, optionally attached to a support.

In this regard, a further object of this invention resides in a method of detecting the presence or the risk of developing an encephalopathy in a subject, the method comprising (i) providing a biological sample containing proteins (or fragments thereof) from the subject, (ii) contacting said sample with at least an antibody as defined above, and (iii) determining the presence of antibody-antigen complexes, the presence of such complexes indicating the presence or the risk of developing an encephalopathy in the subject.

The present invention also concerns kits for the implementation of the aforementioned methods. The kits of the invention more generally comprise a nucleic acid molecule or antibody as defined above, or a nucleic acid array as defined above, or a nucleic acid preparation or library as defined above. The kits may further advantageously comprise control clones for calibration of the detected signals.

A specific object of this invention thus resides in a product comprising, immobilised on a support, at least one specific target molecule selected from a nucleic acid molecule, a vector, a polypeptide and an antibody as defined above. The support may be of various shapes, nature and origin, such as a filter, a membrane, a slide, a polymer, a glass, a plastic and a biomaterial.

The invention also encompasses nucleic acid arrays comprising at least one nucleic acid molecule or vector comprising the sequence of all or part of a sequence selected from SEQ ID Nos 1-15 or a sequence complementary thereto or a functional equivalent thereof. The array comprises preferably at least two distinct nucleic acid molecules as defined above, more preferably at least 3, even more preferably at least 4. Typically, the array comprises at least 5, more specifically at least 8 of said molecules, or even all of them.

Nucleic acid arrays are preferably comprised of a nucleic acid molecule attached to a support, such as a filter, membrane, slide, polymer, glass, plastic, biomaterial, etc. The support may be flat or not, solid or semi-solid. It includes beads, etc. Such DNA-chips or oligo-chips are also included in the instant invention. They can be prepared according to known techniques (see WO99/46403).

The invention also encompasses methods of selecting candidate drug compounds comprising contacting a test compound with a target selected from a nucleic acid molecule, a vector, a recombinant host cell, a polypeptide and an antibody as defined above, and assessing the ability of the test compound to bind to or to modulate the activity of said target in vitro or in vivo.

The invention also relates to the use of the above nucleic acid sequences as targets in screening assays to select candidate drug compounds. The screening assay comprises, for instance, contacting the target (nucleic acid or corresponding polypeptide or protein) with a test compound and assessing the ability of the test compound to bind to or to modulate the activity of said target in vitro or in vivo.

Binding can be determined by any conventional technique, such as immunoassays, for instance, or binding assays (RIE, ELISA, SPA, FRET, etc.). Modulation of the activity can be assessed in various cellular assays or in a cellular assays, using for instance enzyme substrates, reporter genes, etc.

Other aspects of this invention will be described in the following examples, which should be regarded as illustrative and not limiting.

### LEGEND TO THE FIGURES

FIG. 1: Identification of spleen markers associated with TSE infection

FIG. 2: Expression pattern of a genetic marker of TSE

FIG. 3 Identification of circulating markers using a Macro-array.

### EXAMPLES

### Example 1. TSE Markers Obtained in an Experimentally Infected Mouse Model

C57BL/6 mice were either intra-cerebrally or intra-peritoneally infected with brain homogenate containing the murine C506M3 strain derived from a natural case of sheep Scrapie. Control mice were inoculated with brain homogenate of healthy animals. At different time points before and at clinical appearance (i.e., pre-and post-symptoms) diseased animals were sacrificed and total RNA of brain and spleen were prepared. Tissue samples were collected at 35, 70, 111, 148, 190 and 230 days after intra-peritoneal inoculation, whereas tissue collection was performed at 28, 63, 93, 121, 135 and 153 after intra-cerebral inoculation.

Brain samples were studied to identify genes involved in brain invasion and neurodegeneration.

Spleen samples were also evaluated since the PrpSc propagation is dependent on the immune system and is noticeably present in the spleen follicular dendritic cells. The sequences identified from spleen samples are thus providing information on the mechanisms involved in PrpSc propagation through the immune system. The signatures obtained from spleen represent the repertoire of qualitative differences that distinguish infected and non-infected situations that can arise in various cell types. Among these cells are the circulating blood cells whose gene expression can be altered by the presence of even low (currently undetectable) amounts of PrpSc. Since this PrpSc may be expressed either in the circulating cell with the altered profile, or in resident non-circulating cells interacting with the circulating cells, it can be envisioned that some of the signatures identified in spleen will be specifically detected in the blood cells of infected animals.

### 1.1 Identification of Potential Markers at Different Time Points After Infection

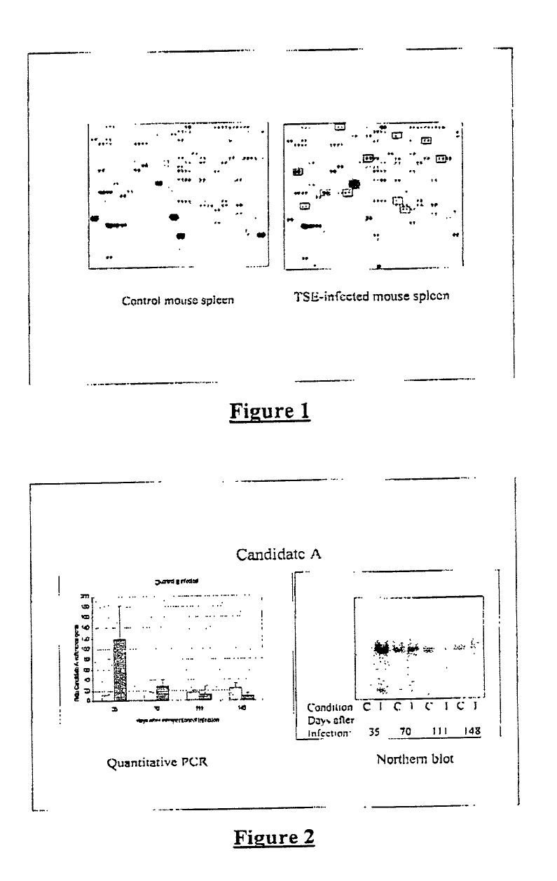

DATAS profiling assays were carried out between pooled RNAs derived from spleen or brain tissue from five infected and five control mice at different stages of the disease. A macro-array containing all DATAS fragments was constructed. TSE infected animals were profiled against control animals by differential hybridisation in order to identify TSE specific signatures. For each time point after infection the macro-array was hybridised with a minimum of two probes derived from control and infected tissue. Z-scores of each clone for each hybridisation were calculated. Statistical z-score analysis identified differentially expressed DATAS fragments with a probability of at least 95% by cross-comparison between the results obtained from the hybridisations with two control and two infected probes. FIG. 1 below shows an example indicating that DATAS can identify spleen markers (outlined in red boxes) that are specifically associated with TSE infection.

Results of z-score analysis are indicated in the table below: numbers <−2 indicate a >95% probability of down-regulation of clones, whereas >+2 reflect an up-regulation with a probability >95%.

Summary table of identified markers

| | | | Z-score |
|---|---|---|---|
| Marker | Probe | Up/down regulation | (pre-clinical stage) |
| A | Spleen | up | 4.10 |
| B | Spleen | up | 3.76 |
| C | Spleen | up | 3.52 |
| D | Spleen | up | 2.31 |
| E | Spleen | up | 3.2 |
| F | Spleen | up | 2.2 |
| G | Spleen | up | 2.7 |

### 1.2. Kinetic Studies of Selected Diagnostic Candidates

Candidates selected by differential hybridisation were further characterised in kinetic studies using quantitative PCR. Expression patterns of three potential candidates in spleen have been established in three infected mice and two control mice. Data were normalised to a reference gene, whose expression is not altered during disease progress. Northern blotting of individual spleen samples derived from two control and infected mice at different times after infection confirmed the expression pattern previously estimated by quantitative PCR. FIG. 2 shows the expression pattern of one candidate determined by quantitative PCR as well as Northern blotting, in each lane 20 μg of total RNA was loaded.

The candidates validated so far were prioritised for analysis based on their cellular localisation. All encode proteins that can be processed and are normally secreted by cells. It is reasonable to assume that their specific up regulation can be detected in blood at the level of RNA in circulating cells or at the level of proteins directly in blood.

### Example 2. Diagnostic Markers Obtained in a Naturally Infected Romanov Sheep Flock

LVK sheep are either naturally infected by or resistant to Scrapie. The symptoms of Scrapie appear on infected animals after 12 months. The first year of life of these animals corresponding to the pre-symptomatic phase. Spleen tissue was obtained from these animals at the age of 6 and 9 months and total RNA of spleen samples were prepared. In addition, total RNA of blood was prepared from Scrapie infected sheep at preclinical and clinical phases.

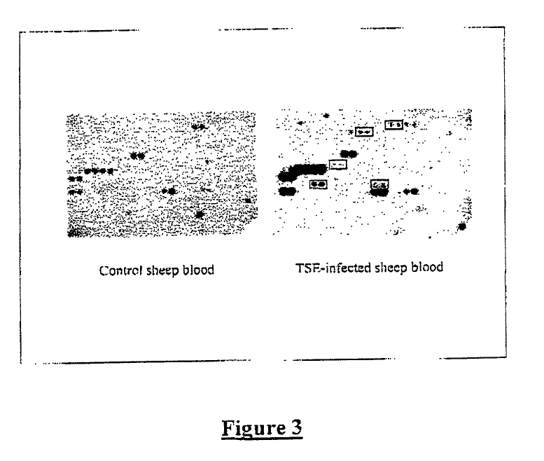

DATAS profiling was performed on RNA derived from spleen and pre-clinical blood samples of infected and resistant/control sheep. The macro-array containing all the DATAS fragments isolated in the sheep model was hybridised with probes derived from blood samples collected from two control sheep, four infected sheep in pre-clinical and clinical phase of the disease. Differential expression was also determined in pooled spleen samples derived from four infected and four resistant sheep at 6 months old. A macro-array challenged with a blood probe is shown in FIG. 3, indicating that we can identify circulating markers.

In total, three candidates were isolated by hybridisation performed with probes derived from blood samples and two by hybridisation with spleen samples. Probing the mouse macro-array with individual sheep blood probes resulted in the identification of two additional candidates. The table below shows the results of the z-scores analysis: numbers <−2 indicate a >95% probability of down-regulation of clones, whereas numbers >+2 reflect an up-regulation with a probability >95%.

| | | | | |
|---|---|---|---|---|
| | | Up/down | Z-score | Z-score |
| Marker | Probe | regulation | Pre-clinical stage | Clinical stage |

Summary table of identified markers hybridising sheep macro-array

| | | | | |
|---|---|---|---|---|
| 1 | Blood | down | −1.9 | −2.5 |
| 2 | Blood | up | 3.4 | 3.9 |
| 3 | Blood | down | −2.1 | −2.3 |
| 4 | Spleen | down | −2.6 | |
| 5 | Spleen | up | 2.8 | |

Summary table of identified markers hybridising mouse macro-array

| | | | | |
|---|---|---|---|---|
| 6 | Blood | down | −2.17 | −1.7 |
| 7 | Blood | down | −2.46 | −2.55 |

For some genes a direct functional link could be established based on literature research. Irrespective of the outcome of the reverse Northern blotting, those potential markers are included in subsequent research.

Eight potential markers are currently under investigation in terms of their expression levels during TSE progress. Furthermore, their expression patterns in blood will be correlated to different polymorphisms of the prion protein known to be associated with the sensitivity of sheep to the Scrapie agent. ExonHit has access to blood samples of sheep with the ARR/ARR genotype, which are resistant to prion disease, with the VRQ/VRQ genotype, which is highly sensitive to Scrapie infection and with the ARR/VRQ genotype, which has an incidence disease of 5%. Furthermore, blood samples from a kinetic study in sheep experimentally infected with the BSE agent are being collected for ExonHit in order to determine their inducibility by the BSE agent of the early-identified markers.

### Example 3. Identification of Circulating BSE Markers at Pre-Symptomatic Stages

Markers identified in the sheep and mouse model can be validated in BSE infected cattle by quantitative PCR and Northern blotting. Macro-arrays containing DATAS fragments isolated in both the sheep and mouse model can be challenged with a panel of blood probes derived from BSE infected cattle at pre-clinical stages in order to identify new BSE specific signatures.

Concurrently with the analysis outlined above, further DATAS experiments using blood samples of BSE infected cattle at different pre-clinical stages in comparison to healthy animals can be performed to identify additional potential markers. The identified candidate markers will be further validated performing quantitative PCR, and added to the final diagnostic if necessary.

A typical diagnostic assay is based on the use of DNA chip, PCR detection or antibody detection.

1. A method of detecting the presence or the risk of developing an encephalopathy in a subject, the method comprising (i) providing a biological sample containing nucleic acids from the subject, (ii) contacting said sample with at least a nucleic acid molecule comprising the sequence of all or part of a sequence selected from SEQ ID Nos 1-15 or a sequence complementary thereto, under conditions allowing hybridisation to occur, and (iii) determining the presence of hybrids, the presence of such hybrids indicating the presence or the risk of developing an encephalopathy in the subject.

2. The method of claim 1, wherein the contacting step comprises contacting the sample with at least a nucleic acid molecule comprising the sequence of all or part of SEQ ID NO:1 or a functional equivalent thereof or a sequence complementary thereto.

3. The method of claim 1, wherein the contacting step comprises contacting the sample with at least a nucleic acid molecule comprising the sequence of all or part of SEQ ID NO:2 or a functional equivalent thereof or a sequence complementary thereto.

4. The method of claim 1, wherein the contacting step comprises contacting the sample with at least a nucleic acid molecule comprising the sequence of all or part of SEQ ID NO:3 or a functional equivalent thereof or a sequence complementary thereto.

5. The method of claim 1, wherein the contacting step comprises contacting the sample with at least a nucleic acid molecule comprising the sequence of all or part of SEQ ID NO:4 or a functional equivalent thereof or a sequence complementary thereto.

6. The method of claim 1, wherein the contacting step comprises contacting the sample with at least a nucleic acid molecule comprising the sequence of all or part of SEQ ID NO:5 or a functional equivalent thereof or a sequence complementary thereto.

7. The method of claim 1, wherein the contacting step comprises contacting the sample with at least a nucleic acid molecule comprising the sequence of all or part of SEQ ID NO:6 or a functional equivalent thereof or a sequence complementary thereto.

8. The method of claim 1, wherein the contacting step comprises contacting the sample with at least a nucleic acid molecule comprising the sequence of all or part of SEQ ID NO:7 or a functional equivalent thereof or a sequence complementary thereto.

9. The method of claim 1, wherein the contacting step comprises contacting the sample with at least a nucleic acid molecule comprising the sequence of all or part of SEQ ID NO:8 or a functional equivalent thereof or a sequence complementary thereto.

10. The method of claim 1, wherein the contacting step comprises contacting the sample with at least a nucleic acid molecule comprising the sequence of all or part of SEQ ID NO:9 or a functional equivalent thereof or a sequence complementary thereto.

11. The method of claim 1, wherein the contacting step comprises contacting the sample with at least a nucleic acid molecule comprising the sequence of all or part of SEQ ID NO:10 or a functional equivalent thereof or a sequence complementary thereto.

12. The method of claim 1, wherein the contacting step comprises contacting the sample with at least a nucleic acid molecule comprising the sequence of all or part of SEQ ID NO:11 or a functional equivalent thereof or a sequence complementary thereto.

13. The method of claim 1, wherein the contacting step comprises contacting the sample with at least a nucleic acid molecule comprising the sequence of all or part of SEQ ID NO:12 or a functional equivalent thereof or a sequence complementary thereto.

14. The method of claim 1, wherein the contacting step comprises contacting the sample with at least a nucleic acid molecule comprising the sequence of all or part of SEQ ID NO:13 or a functional equivalent thereof or a sequence complementary thereto.

15. The method of claim 1, wherein the contacting step comprises contacting the sample with at least a nucleic acid molecule comprising the sequence of all or part of SEQ ID NO:14 or a functional equivalent thereof or a sequence complementary thereto.

16. The method of claim 1, wherein the contacting step comprises contacting the sample with at least a nucleic acid molecule comprising the sequence of all or part of SEQ ID NO:15 or a functional equivalent thereof or a sequence complementary thereto.

17. The method of claim 1, wherein the nucleic acid is immobilized on a support, such as a chip, filter, membrane or a glass slide.

18. The method of claim 1, wherein the biological sample comprises blood, serum, saliva, urine, a tissue sample or a cell sample, preferably blood.

19. A nucleic acid molecule selected from the group of SEQ ID Nos 1-15 or a fragment thereof, a sequence complementary thereto or a functional equivalent thereof.

20. A vector comprising a nucleic acid of claim 19.

21. A recombinant host cell comprising at least one nucleic acid molecule of claim 19 or vector of claim 20.

22. A nucleic acid array comprising at least one nucleic acid molecule of claim 19 or vector of claim 20.

23. A polypeptide, wherein said polypeptide has an amino acid sequence encoded by a nucleic acid molecule of claim 19.

24. An antibody that binds a polypeptide of claim 23.

25. A product comprising, immobilised on a support, at least one specific target molecule selected from a nucleic acid molecule of claim 19, a vector of claim 20, a polypeptide of claim 23 and an antibody of claim 24.

26. The product of claim 25, wherein the support is selected from a filter, a membrane, a slide, a polymer, a glass, a plastic and a biomaterial.

27. A method of selecting candidate drug compounds comprising contacting a test compound with a target selected from a nucleic acid molecule of claim 19, a vector of claim 20, a polypeptide of claim 23 and an antibody of claim 24, and assessing the ability of the test compound to bind to or to modulate the activity of said target in vitro or in vivo.

28. A method of detecting the presence or the risk of developing an encephalopathy in a subject, the method comprising (i) providing a biological sample containing proteins from the subject, (ii) contacting said sample with at least an antibody of claim 24, and (iii) determining the presence of antibody-antigen complexes, the presence of such complexes indicating the presence or the risk of developing an encephalopathy in the subject.

29. The method of claim 1, wherein the subject is a mammal selected from a cow, sheep or a goat.


SEQUENCE LISTING (15 sequences)

SEQ ID NO: 1 - DNA, 318 bp, mammalian
ccccagagaggagccgaagctcacgatgccatgcacctgccactggccattagatgcccggcaattcagtggtccgccagagtccccattgcagctggaggtcacgccgtcgccaccagcgcacaccatgctggacttcacagagcttccccaccagctagcgctggagcaggtggcatagtccacaaccagcaggcggccctgcctcagggtgtcaggactgttcccattggtctgcagcaggccccagcctgtgacatagcagacatagtttctcgggagaatggtgccagcgggtgggaggcaagctgtctggat

SEQ ID NO: 2 - DNA, 224 bp, mammalian
atggaccaggggtagaacaacttgctgcaggcgagctggtgtcacattgcccacaccactgattcggccccagccagtggtgacacaggtgagccccgaaggcagtgcctcgtttgtggaagccaggcagactggtgagacttgtgctgtgtaccgggctggcgaggcaagcttcaggagagtcaggtcattgttcatggtgttggcgttccagttagggtggc

SEQ ID NO: 3 - DNA, 276 bp, mammalian
cggtggcaagtcgggttccaggtccatgaagcccccgggaggagaatcgagcgatcttttcggaagtccagaagaaggtatttcttcaagcaagcctaataggatggcatctaatattttcggaccaactgaagaacctaaaaacatacccaagaggacaaatcctccaggaggcaaaggaagtgggatctttgatgaatcgactcctgtgcaaactcgacaacgtttgaatccaccaggggggaagaccagtgacatatttgggtccccagtcct

SEQ ID NO: 4 - DNA, 303 bp, mammalian
caacagacacagtctcagagaactgggcaggagtggccagcttcagcagggtgatgtcattacgcacggtgaaggagttgaacttggggttcttaaaaacctgagcgattttcaggacctggacattctcttcgtcggagccctgatcaaactctccagctaccaccacatcggttgtcttgaccccgcagtgggcagcagtgaccacccagttttcgctgatgagggagcccccgcagaaatggaagccagttctgtcctgcagggacacctgccagggccaggagccagggatagcatcct

SEQ ID NO: 5 - DNA, 130 bp, mammalian
  variation (101): A, T, G or C
gtcttggtggtccaatccagtcattctgatcttttccttctggaaatttctattccaacggtaactcgacattactcttgtgaatccataaggatgagccntcataaatccgacagccattttatacatt

SEQ ID NO: 6 - DNA, 266 bp, mammalian
gggtggcctcgaactcagaaatccgcctgcctctgcctcccgagtgctgggattaaaggcgtggccaccacgcccggctttgcatgctttatttcttgtggaataactgacacccaagttctccttcagaagcttcagccaagcccaccttgaggaacaagacgaggacacatgatgggtgagacatggcagaggtcctggcggcacggcccagttccccggcatctcctcccacaggccagctacttattcagggacagcgactg

SEQ ID NO: 7 - DNA, 362 bp, mammalian
cgtggacgatggctaagtaccggtctacagctatacaggccagcagcaggctgctgcagtagaaattgatcttgtgcagagcgatcacagttttgcagaggaaggtccctaggacccaacccacagagccctcagccactgcaaaaggcaggatgaagactaagagaaggtcggctactgcgaggtggaacaggaaggtctcggttgagctccgcgtgtgccggtgcctctccgggattccagcaccaggatgtttcccatcatacccaggaggaagatgaggctgtaggcccaggcatgaatccgccttaaggacgtcagtaagggtccctcgactgtagagcagaagttctgtctgtagg

SEQ ID NO: 8 - DNA, 320 bp, mammalian
ggatgcatttgaactgataaggactaggtagaactgaagggctagatggaatgttacggcctaggtataacgttaagcctaagtaactcttacgtggctagcctgccattttgcgctgttactagtattataaggaaacttccttatgtgcaagttgattgcatattctcttaaattctttgctcttggaaactgagcacaacagaggttagttagaactgctctgtatagttagccaaaatgagctttgacccaatcagccaatcagcagcacttctgcatatgtgtaaagcttgtatggtatctgcttttataagctg

SEQ ID NO: 9 - DNA, 220 bp, mammalian
gtgtccaggagggaactggtatgatctaatgaatccttttactaagatggggatgtgatggtagcacacagcagggaagagggacttcgaatctcaggcctcagcttagaaggggaagcacctatttccactgccccttctttaagacatctcccttttgctgaggcttaccagggggtaggggagcgcagggaaggtcaaggaggtgtatcaaagtatc

SEQ ID NO: 10 - DNA, 224 bp, mammalian
agacactttgatacacctccttgaccttccctgcgctcccctaccccctggtaagcctcagcaaaagggagatgtcttaaagaaaggggcagtggaaataggtgcttcccctcctaagctggggcctgagattcgaagtccctcttccctgctgtgtgctaccatcacatccccatcttagtaaaaggattcattagatcataccagttccctcctggacaccc

SEQ ID NO: 11 - DNA, 197 bp, mammalian
taccatgagggagtggctggattaggcctagggaggatgactgtccatgagagatgacaggtgtgggcagctcttctagggggtgtgggcactggagtagcctcaggaggcagcggctcccccgctgttggttctgagactggtgaggcgggaccagccccgttgtttccagttcttcatgcctggtggcaccctca

SEQ ID NO: 12 - DNA, 143 bp, mammalian
gggtaaaagagggaaatgaaaaggagagagacagtatccagctcggtaaacagtttccctaagtgttctccaccatgtggaacacacaggagattcatgggagttgggtagagaagagaagggggaaggaggagacagaggca

SEQ ID NO: 13 - DNA, 310 bp, mammalian
ctgacccagatcacagcctgggctggtacccagtggggcgaattcagatgagtaacacggaggaccctccgtggacacctggcaaaaatctatgccatgcactgggggacagactcaaggctgctggtcagcgcctcccaggacggaaagctcatcatttgggacagctcaccactaacaaggtccacgccatccctctgcgttcctcctgggtaatgacctgtgcctcgccccctcagggaactttgtggcctgtgggggtttggacaacatctgctccatctatagtctcaagacccgagagggcaat

SEQ ID NO: 14 - DNA, 306 bp, mammalian
tcgacccccgcctgtttaccaaaaacatcacctctagcattacaagctattagaggcactgcctgcccagtgactaaagtttaacggccgcggtatcctgaccgtgcaaaggtagcataatcacttgttccttaattagggactagcatgaacggctaaacgagggtccaactgtctcttatctttaatcagtgaaattgacctttcagtgaagaggctgaaatataataataagacgagaagaccctatggagctaaattatataacttatctattaatttataaacctaatggcccaaaactat

SEQ ID NO: 15 - DNA, 303 bp, mammalian
tggctgtgtcccagttgtcattgcagactcttatattctgcctttctctgaagttctggactggaagagggcatctgtggtcgttccagaggaaaagatgtcagatgtgtacagcatcctgcagaacatcccacagaggcagattgaagagatgcagagacaggcacggtggttctgggaggcatacttccagtccattaaagccattgccctggccccctacagatcatcaatgacaggatctatccatatgcagcctctcctatgaagagtggaatgaccctcctgctgtgaagtgggcta